(19) United States
(12) Patent Application Publication
(10) Pub. No.: US 2024/0174981 A1
(43) Pub. Date: May 30, 2024

(54) METHODS FOR THE FABRICATION OF 3D-STRUCTURED EDIBLE FAT USING PLANT-BASED SCAFFOLDS

(71) Applicant: Cultimate Foods UG, Berlin (DE)

(72) Inventors: Mariia Abyzova, Hanover (DE); Amir Zakirov, Göttingen (DE)

(73) Assignee: Cultimate Foods UG, Berlin, BE (DE)

(21) Appl. No.: 18/518,774

(22) Filed: Nov. 24, 2023

Related U.S. Application Data
(60) Provisional application No. 63/427,937, filed on Nov. 25, 2022.

(51) Int. Cl. C12N 5/0775

(52) U.S. Cl. CPC C12N 5/0667; C12N 2533/90

(57) ABSTRACT

3D-structured fat compositions and methods are disclosed, wherein the compositions and methods optimize and/or use one or more of parameters that make the fat more cost-effective yet more closely mimicking wild-type fat (i.e. from traditional animal farming). The compositions and methods optimize sterilization protocols; select and identify the ideal plant-based matrix to be used; identify, isolate, and proliferate the ideal cells; identify and use serum, or alternatively, use minimal or no serum, and media supplements; use one or more methodologies to enhance the ideal cells proliferation; and develop a quantitative non-invasive method for ascertaining the ideal cells' proliferation and/or adhesion.

### CLAIM OF PRIORITY

This application is a Non-provisional application claims priority from U.S. provisional Application No. 63/427,937 filed Nov. 25, 2022 the contents of which is hereby fully incorporated by reference.

### FIELD OF THE INVENTION

The present invention relates to compositions and methods for fabricating 3D-structured fat using plant-based scaffolds. The various parameters that go into the production of this 3D-structured fat are evaluated herein to generate the best possible product using these plant-based scaffolds.

### BACKGROUND OF THE INVENTION

Animal agriculture is associated with a host of ever-increasing environmental and public health issues. The increase in close human-animal contact from animal agriculture, destruction of wildlife habitats, and rising human population and global mobility means that about 75% of new infectious diseases in humans arise from zoonotic sources (i.e., transmission from animals to humans). Animal farming on an intensive scale also plays a substantial role in antibiotic resistance, as approximately 80% of all antibiotics sold in the United States and approximately 70% of antibiotics sold globally are administered to livestock. Much of the deforestation of sensitive habitats such as the Amazon rainforest, which irreversibly threatens biodiversity and brings humans close to displaced wild animals, is due to livestock grazing or feed cropping. A 2020 report from the UN Environment Programme on preventing future pandemics noted that two of the seven major anthropogenic causes of zoonotic disease are increased global demand for animal protein products and unsustainable agriculture intensification, such as the rise of intensive animal agriculture in places that were traditionally forested.

Moreover, greenhouse CO₂ gas emissions from livestock represent roughly 15% of human-caused emissions. In addition to climate impacts, meat production comes at a high environmental cost in areas such as land and water use, primarily due to the aforementioned gas emissions, animal feed production and animal waste contamination. Public concerns around climate change appear to drive purchasing behavior at least to some extent, as increased media coverage of climate change is associated with decreased demand for beef However, global meat consumption continues to rise steadily despite increased public awareness of climate change, suggesting that the desire to avoid climate impacts is, by itself, not sufficient to meaningfully curb meat consumption.

One pragmatic means of possibly addressing the negative externalities of meat production is by changing the production process rather than targeting large-scale consumer behavior change. Cultivated meat (CM; also called cell-based or cultured meat) is meat grown from animal stem cells, mimicking the process by which cells grow and divide in vivo. The goal is to produce a product with the same nutritional and organoleptic properties as its conventional counterpart, and the technology is continually improving. The first experimental demonstration of CM was reported in 2002, which showed that cultured fish cells could contribute to the growth of a goldfish muscle explant. The first reported tasting of CM occurred in 2013 with the much-publicized hamburger produced by Dr. Mark Post's team. Today, a growing number of companies (at least 100 as of mid-2023) are working to commercialize and scale CM. The first regulatory approval for cultivated chicken occurred in December 2020 in Singapore, with commercial sales following shortly thereafter.

One drawback of the currently available CMs is the inability of the in vitro differentiating stem cells to adequately mimic all of the types of cells and their specific properties that are typically found in wild-type meat (i.e. from traditional animal farming). This means that the CM, although full of protein, does not have the structural characteristics, texture and density, and taste present in its wild-type counterpart. To properly develop CM that has the consistency and taste present in wild-type meat, one needs to make CM products that have the correct proportions of cell types present in the natural meat, including fat cells. Fat is a key determinant of flavor, texture, nutrition, and visual appearance correlated with consumer preference. Cultivated fat (CF, i.e. cell-cultivated fat cells) can be used as novel ingredients combined with other ingredients from plants, microbes, or animal cells to perfectly mimic conventional animal-based products. These hybrid products have the potential to unlock the consumption of plant-based meat and pave the way for cell-cultured meat. However, current strategies are insufficient to produce these cell-cultivated products with various cell types. To achieve this, one needs to better understand the involved parameters and the optimal way to perform the multi-step CF product. Moreover, developing the method steps with plant-based scaffolds is an area in which improvement is needed. It is with these needs that the current invention was developed.

### BRIEF SUMMARY OF THE INVENTION

The present invention relates to compositions and methods of using plant-based scaffolds to fabricate 3D-structured fat. In an embodiment, the present invention relates to finding methods of fabricating a fat product that more closely mimics the structural characteristics, texture and density, of its wild-type counterpart (i.e. from traditional animal farming) while using improved methods to generate not just a better product but better yields of the product.

### BRIEF DESCRIPTION OF THE SEVERAL VIEWS OF THE DRAWING

FIG. 1 shows the rate of degradation (in %) of the tested matrixes under the influence of enzymes collagenase and trypsin.

FIG. 2 shows the cell viability (normalized) of cells after being seeded on various plant-based matrixes.

FIG. 3 shows the cell proliferation index (in %) of cells grown in serum-free medium (E8) and other modified mediums (DMEM/F 12 and DMEM+5% FBS).

FIG. 4 shows a graph comparing the rate of cell proliferation over time (number of cells over days) when cultured using a standard medium (DMEM/F 12) and a serum-free medium without TGF β1 (E8−TGF β1).

FIG. 5 shows a graph comparing the efficiency of cell proliferation over time (number of cells over days) when cultured using a standard medium (DMEM/F 12) and a serum-free medium with glutathione supplementation (E8−TGF β1+glutathione).

FIG. 6 shows a graph evaluating the cell growth efficacy (normalized) of cells cultured using a standard medium with 10% FBS (DMEM/F 12+10% FBS) and a modified serum-free medium without insulin and TGF β1 but with the addition of IGF-I (E8+IGF-I−TGF β1).

FIG. 7 shows a graph showing cell adhesion (in %) of plant-based matrixes under different treatments (control, treatment and alginate).

FIG. 8 shows a graph of absorption spectra of iron lactate and iron chloride in DMEM/F 12 culture medium in a microplate analysis.

FIG. 9 shows a graph with the optical density measured from lactate produced from different numbers of cells.

### DETAILED DESCRIPTION OF THE INVENTION

The present invention relates to compositions and methods of using plant-based scaffolds to fabricate 3D-structured fat. In an embodiment, the present invention relates to finding methods of fabricating a fat product that more closely mimics the structural characteristics, texture and density, of its wild-type counterpart (i.e. from traditional animal farming) while using improved methods to generate not just a better product but better yields of the product.

A number of parameters were evaluated to ascertain what level of those parameters gives the optimal results for the fabrication/growth of stem cells to generate fat using a plant-based scaffold. Initially, nine commercially available plant-based scaffolds were evaluated to ascertain which plant-based scaffold has the ideal characteristics for fabricating 3D-structured fat.

### Plant Scaffolds for Cell-Cultivated Fat

The ability to proliferate a C2C12 cell line was evaluated on nine commercially available plant-based scaffolds. Various hydrolysates of soy protein (flour) were served as a substitute for serum and were chosen given their studied use as an extracellular matrix. It was understood that during the cell cultivation process, peptide fragments will be released from this matrix under the influence of proteases, which can positively affect cell proliferation. Consequently, the percentage amount of serum in the used culture medium can be reduced leading to lower culture medium costs.

Nine different matrixes were evaluated:
A—Textured Vegetable Protein (soybean flour), elongated pieces with an approximate size of 4.5-5×0.7-1.0×0.5-0.8 cm
B—Textured Vegetable Protein (soybean flour), irregular pieces approximately 1.2×1.2 cm
C—Textured Vegetable Protein (soybean flour), irregular pieces approximately 2.0×4.0 cm
D—Textured Vegetable Protein (soybean flour), flat pieces approximately 1.0-2.0×7.0-8.0 cm
E—Textured Vegetable Protein (soybean flour, wheat gluten, soybean protein isolate, food salt), imitates irregular pieces of dried meat
F—Textured Vegetable Protein (deodorized low-fat soybean flour, wheat gluten, soy protein isolate, food salt), imitates irregular pieces of dried meat
G—Textured Vegetable Protein (soybean protein isolate, potato starch, calcium carbonate, distilled monoglycerides), granules approximately 3 mm in size
H—Textured Vegetable Protein (natural raw yellow pea), granules approximately 3-5 mm in size
I—Textured Vegetable Protein (soybean flour), granules approximately 4-5 mm in size

### Sterilization of the Matrixes

First, each of the matrixes were evaluated for their ability to withstand sterilization. For sterilization, eight of the matrixes were washed in distilled water followed by autoclaving without liquid for 60 minutes at 121° C. and 1.1 bar pressure. Since sample “G” did not withstand this treatment, this sample was autoclaved for 40 minutes without washing. After the sterilization process, all of the matrixes were incubated in a nutrient-full medium from 24 to 48 hours, no disturbances nor external changes in the structure were noted, and bacterial contamination was absent. All of the matrixes, except sample “G” were considered sterile and they were able to withstand the sterilization procedure indicating that any of the matrixes would pass this initial test. Although sometimes acidification of the medium was noted, additional washing in fresh medium achieved an optimum pH, meaning that any commercially available matrixes could be used.

### Hydrophilicity of the Matrixes

The absorption of water by matrixes [using a testing procedure described by Javier Enrione, 2017] allows us to assess the hydrophilicity of the material. Hydrophilicity is an important parameter to measure since it is directly related to the bitterness of the matrix (the greater the hydrophobicity, such as the hydrophobic side chains of amino acids, the greater the bitterness it will have [Ney, K. H., 1979]). Also, the level of water absorption affects other physicochemical parameters, such as the stiffness of the matrix, the ability to create suitable conditions for cultured cells, and so on.

The matrixes were evaluated for their hydrophilicity by being weighed, and subsequently, incubated in dH₂O for an hour. They were then lightly dabbed with a napkin to remove excess moisture, after which the weighing procedure was repeated. The calculation of moisture absorption was carried out according to the formula (M(wet)−M(dry))/M(dry).” A summary of the results of the study is presented in Table 1

TABLE 1
Dry and Wet weight of the nine matrixes
and their water absorption coefficient.

| Sample | Dry, g | Wet, g | Coefficient of water absorption |
|---|---|---|---|
| A | 1.6 | 4.41 | 1.8 |
| B | 0.3 | 1.00 | 2.21 |
| C | 2.0 | 10.41 | 4.12 |
| D | 2.4 | 6.54 | 1.7 |
| E | 1.4 | 3.75 | 1.64 |
| F | 1.5 | 3.57 | 1.39 |
| G | 0.03 | 0.22 | 7.96 |
| H | 0.05 | 0.20 | 2.88 |
| I | 0.09 | 0.29 | 2.21 |

The data obtained allowed the inventors to identify the most hydrophilic carriers. The most hydrophilic carriers were found to be “C” and “C”. “C” placed in dH₂O for a long period of time showed stability whereas “G” dissolved, forming a solution. However, G is stable in culture media and buffer solutions. All of the remaining samples had approximately the same hydrophilicity and they were all stable in dH₂O.

### Biodegradation and Bioavailability of the Matrixes

The nine matrixes were evaluated for their ability to undergo or withstand biodegradation by gastrointestinal enzymes (as they are used as the scaffold to generate fabricated fat and they will be eaten) and bioavailability. The analysis of the degradation (or splitting) of the matrixes and the transition of their components into solution was carried out after the centrifugation and drying of the sediments produced by the experiments in a drying oven for 120 minutes. Subsequently, the sediments were weighed and the percentage of the matrix that passed into the solution was calculated.

The study of possible biodegradation by gastrointestinal enzymes was carried out on the example of commercial preparations by exposing the matrixes to “Acidin-pepsin” (0.04 mg/ml) (betaine hydrochloride and pepsin) and “Micrazym” (Pancreatin granules containing pancreatic enzymes) (10 mg/ml) to simulate the process of “digestion”. As a result, in the exposure of the matrixes to Acidin-pepsin, visible degradation of the structure of the matrix was observed in samples “F” and “H”, but the other 7 samples out of 9 did not reveal any external changes (data not shown). When exposed to Micrazym, a complete degradation (>40%) was observed for the samples: “B”, “G” and “H”. The remaining matrixes all showed partial degradation of their structure.

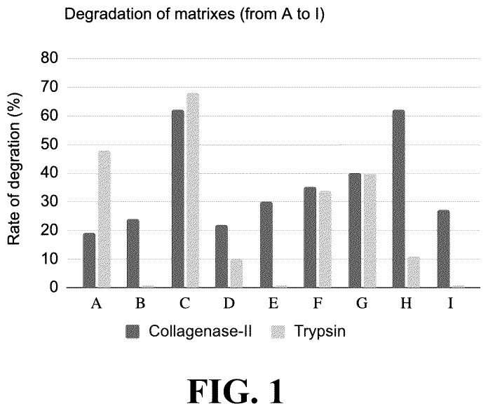

The bioavailability of the matrixes, and the possibility of replacing these matrixes with cell-produced extracellular matrix ones, was studied by treating them with collagenase-II (0.01 mg/ml) and trypsin solution (2.5 mg/ml). The results are shown in FIG. 1. It was noted that matrixes “C” and “G” underwent complete degradation (>40%) under the influence of both collagenase-II and trypsin. The structures of matrixes “B”, “E” and “I” were practically undisturbed under the influence of trypsin.

As can be seen from the graph, the matrixes are more susceptible to the effects of collagenase-II than trypsin except for matrixes “A” and “C”. However, it is possible to distinguish “A” and “C” from the other tested samples, in which, apparently, there are more specific sites for the degradation of the matrix by trypsin. It is known that trypsin cleaves peptides on the carboxylate side of lysine and arginine residues in a peptide. Stated differently, trypsin is known to cleave peptide bonds with sequences -Lys-X- and -Arg-X-, where X may be proline or any amino acid that does not contain acid residues or positively charged groups [see Darbre A., 1989]. Without being bound by theory, the presence of arginine also plays a positive role in cell adhesion. The main hypothesis is that in matrixes “A” and “C”, a glycine residue is located near the arginine residue, as well as an aspartic acid residue, thereby creating the ensemble “RGD”, and thus promoting cell adhesion. However, the presence of aspartic acid (D) in this sequence is dubious as it has been reported that trypsin would not break down the structure with an aspartic acid residue [Dong-an W., 2002, Nakajima K., 1990]. However, it is postulated that glycine is present with the hydrophobic amino acids, leucine and isoleucine, since the matrix is affected by collagenase, and collagenase is known to have a specific cleavage site that includes the Gly-Ile or Gly-Leu bond [see Wu H, 1990]. Therefore, matrixes “C”, “F” and G” may be of the most interest in terms of a time frame for using cells, which are equally degraded by both enzymes and, presumably, can be replaced by a newly synthesized extracellular matrix.

The cytotoxicity of the enzymatically generated hydrolysates of the scaffolds and their effects on C2C12 line cell proliferation was evaluated by an MTT test [Präbst, K., 2017]. MTT or 3-(4,5-dimethylthiazole-2-yl)-2,5-tetrazolium bromide is capable of ascertaining the metabolic activity of cells by measuring the metabolic activity of transforming the water-soluble MTT into formazan by the mitochondria of living cells. Accordingly, the assay test for proliferation abilities at given concentration levels. The assay used an effluent solution concentration of 5 mg/ml of MTT in PBS at pH 7.3, with a working solution concentration of 0.5 mg/ml. The cells were seeded in a 96-well plate at a concentration of 12500 cells/ml, and after 24 hours the medium was replaced by fresh medium with a reduced serum content of down to 5%.

Based on the obtained data, the hydrolysates after collagenase-II treatment do not show pronounced toxicity. The hydrolysates of matrixes “A”, “B”, “C”, “D”, “E” and “F” created after the treatment with collagenase-II have a stimulating effect on proliferation, about 50%-70% relative to control (untreated cells), which means that the concentration of serum can be reduced from at least about 5% to 10%. In contrast to the other samples, it was demonstrated that matrixes “G”, “H” and “I” showed a slight inhibition of cell growth, releasing low-toxic fragments.

On the other hand, the hydrolysates obtained under the influence of trypsin practically do not cause an increase in proliferation; on the contrary, they lead to a slight inhibition of growth. The greatest effect of trypsin is shown by samples “A” and “B”, which inhibit cell growth by about 45-40%. Specifically on matrix “B”, not only did the matrix show inhibited cell growth under the influence of trypsin, but its structure did not significantly degrade (FIG. 1). Without being bound by theory, it is believed that this can be explained by the fact that matrix “B” is still exposed to the enzyme and secretes toxic peptides. It is worth noting that at low concentrations of hydrolysates from treating matrixes “A”, “B”, “C”, “E” and “F”, with collagenase-II a slight increase in cell proliferation was demonstrated, which indicates that these matrixes may be ideal candidates for use and/or for further study in cell cultures.

Subsequently, the adhesion of cells on the various matrixes was tested.

### Adhesion of Cells on Matrixes

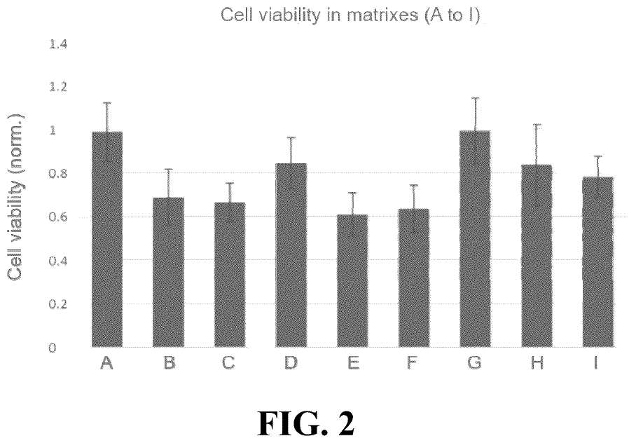

The prepared fragments of the matrix were laid out in the wells of the 24-well plate. Cells were seeded on a matrix at the rate of 10⁵ cells/ml. A control was prepared in which the same number of cells were sown to the bottom of the well. After 24 hours, the survival abilities of cells on the surface of the matrixes were investigated. All of the matrixes and cells were placed in a fresh culture medium containing MTT (0.05 mg/ml) with incubation occurring for 2 hours at 37° C. in a CO₂ incubator. The results are shown in FIG. 2.

FIG. 2 shows cell survival in the various tested matrixes. The greatest affinity of cells to the matrix was found to occur for samples “A”, “D”, “G” and “H”. The obtained data from the samples make it possible to assess the degree of cell adhesion and the possibility of cultivating cells on these matrixes. It is believed that for matrixes “B”, “C”, “E”, “F” and “I”, additional surface treatment with components that increase adhesion, such as the addition of gelatin or the introduction of RGD segments, might prove beneficial. In any event, it should be noted that all matrixes demonstrated cell survival that exceeded 50%, and the matrixes “A” and “G” demonstrated results that were comparable to the stem cell control (the adhesion of culture plastic).

Subsequently, the influence of serum in the culture media was studied to ascertain the factors that would give the best results. The focus of the study was to reduce or potentially eliminate the serum so as to reduce costs.

### Developing a Serum-Free Environment

A review of the literature and the tests enumerated herein found that certain combinations of growth factors to support cell viability and proliferation should be studied to develop a serum-free medium. Initially, the E8 composition was used as a starting point to evaluate potential serum-free media [Guokai Chen et al., 2011].

The composition of E8 is a modification of the Dulbecco's modified Eagle's medium and is shown in Table 2. PGP-22 T1

TABLE 2
Composition of E8 medium.

| DMEM/F12 | Base | |
|---|---|---|
| L-Ascorbic Acid 2 Magnesium Phosphate | 64 | mg/l |
| Sodium selenite | 14 | μg/l |
| Transferrin | 10.7 | mg/l |
| Sodium bicarbonate | 543 | mg/l |
| Insulin | 19.4 | mg/l |
| FGF2 (fibroblast growth factor 2) | 100 | μg/1 |
| TGF β1 (transforming growth factor - β1) | 2 | ng/ml |

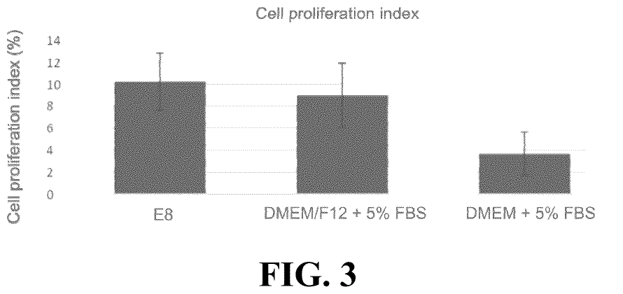

To assess the effectiveness of this medium, ADSC p4 (passage 4) were seeded in a six-well plate at a concentration of 1×10⁵ cells, as a control for E8 (Gibco Essential Medium), the classical DMEM/F12 and DMEM with 5% FBS (fetal bovine serum) was used. After 48 hours of incubation, the number of cells was counted and the cell proliferation index was calculated. The results are shown in FIG. 3. The proliferation index of cells seeded on E8 is statistically different to the DMEM/F12 and DMEM containing 5% FBS.

However, because the E8 medium has TGF β1 (transforming growth factor β-1) in its composition, which is a factor that can potentially affect the further differentiation of ADSC/MSC cells further study was warranted. In this regard and for comparison purposes, it was decided to study the behavior of cells in an environment that does not contain transforming growth factors. To perform this study, the cells were cultured over 5 hours in the E8 medium with no TGF β1 in a six-well plate and compared to the standard DMEM/F12 medium, which was used as a control. After 5 hours, the cells were removed using trypsin, counted and seeded again in the same concentration of 20×10³ cells into the well.

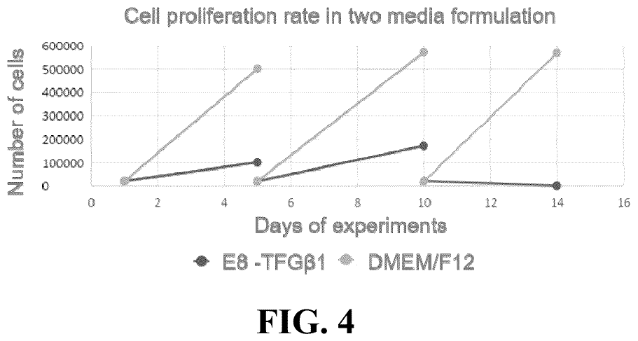

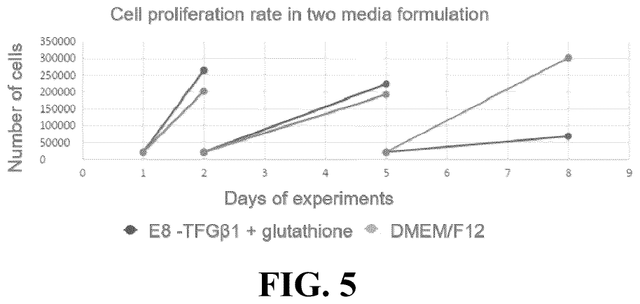

FIG. 4 shows a graph comparing the rate of cell proliferation over time when using a standard medium and a serum-free medium, with no TGF β1 (E8−TGF β1). As can be seen from the graph, the absence of TGF β1 leads to a decrease in the rate of proliferation followed by cell death. According to the literature, the presence of serum in the culture medium can affect cellular adhesion, as well as maintain the antioxidant potential of the cell. To test these parameters, glutathione was added as a composition in the absence of serum as it is known to be an antioxidant. Glutathione was added at an effective concentration of 0.04 mg/ml and the metabolic rates were measured by the MTT (3-(4,5-dimethylthiazol-2-yl)-2,5-diphenyltetrazolium bromide) test method. The results of this experiment are shown in FIG. 5.

FIG. 5 shows a graph comparing the efficiency of cell proliferation on a standard medium and a serum-free medium with glutathione supplementation. The results of FIG. 5 confirm that adding glutathione to the medium not only supports cell proliferation but also accelerates it. However, the addition of glutathione reduces the shelf life of the medium used due to the low stability of this compound, which ultimately leads to a slowdown in cell proliferation.

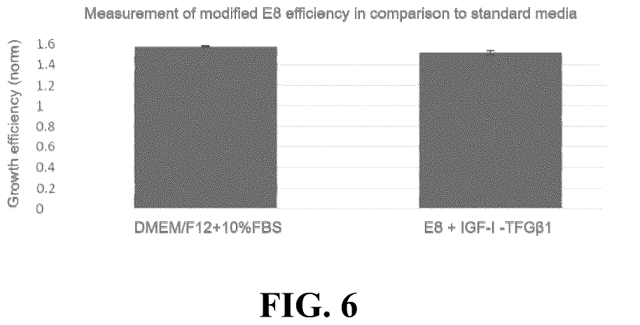

Insulin-like growth factors (IGF-I and IGF-II) have been shown to play roles in the inhibition of cell death, and the promotion of cell proliferation and differentiation. While IGF-II positively influences the myogenic differentiation of myoblasts through three signaling pathways depending on the redox potential of the cell, IGF-1 stimulates the proliferation of pre-adipocytes and promotes their differentiation into mature adipocytes. Moreover, IGF-I has structural and functional similarities to insulin, which promotes fat storage in cells, and it can interact with insulin receptors even counteracting these effects. Based on this, insulin in the composition of the modified E8 medium was replaced by IGF-I. The effectiveness of this medium was measured and assessed using the MTT reagent. The cells were plated in a six-well plate at a concentration of 100×10³ cells/well, the standard DMEM/F12 medium with 10% FBS was used as a control. As soon as the cell confluence in a well reached a monolayer, the cells were washed and poured with a medium containing an MTT reagent. After 2 hours of incubation, the detection was carried out on an ELISA (enzyme-linked immunosorbent assay) spectrometer at a wavelength of 560 nm. FIG. 6 shows an evaluation of the efficacy of the modified E8 medium without insulin and TGF β1 with the addition of IGF-I (E8+IGF-I−TGF β1). The results using the modified E8 medium to maintain proliferation and subsequent differentiation of ADSCs were statistically significantly not different from the control. This is a positive indication to use the serum-free modified medium.

### Increased Cell Adhesion to Soy Matrix

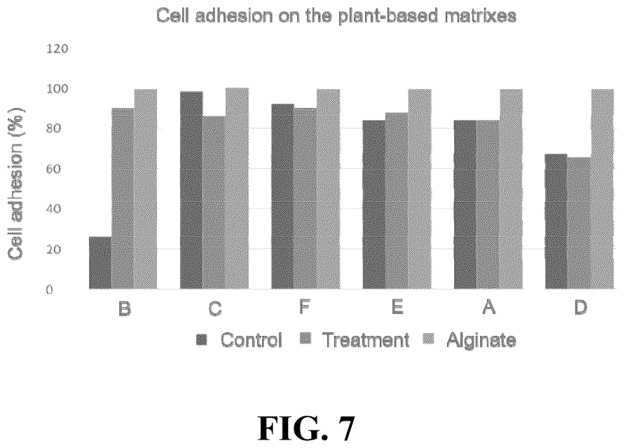

The treatment of the matrixes to a certain mix of enzymes exposes amino acids present in the whole polypeptide chain and increases cell adhesion. Experiments using various enzymes at given concentrations and/or for given durations were tested. Initially, the matrixes were immersed in a collagenase solution with a concentration of 2 μg/ml and incubated for 20 minutes, followed by a three-fold washing using PBS. A culture medium with 10% serum to inactivate the enzyme was also added. Control matrixes were immersed in PBS followed by washing and incubation in a culture medium. The C2C12 cells were seeded on treated matrixes at a concentration of 200,000 cells per matrix. As an analogy with 3D printing technology, some of the cells were planted on the matrix as part of an alginate gel followed by polymerization in a calcium chloride solution (at a 55 mM concentration) for 5 minutes. After an hour of incubation, the matrixes were washed with PBS and the percentage of settled cells was calculated: 200,000 cells were taken as 100%, the entire medium and PBS in 15 ml aliquots were collected from the wells, and after centrifugation, the precipitate was re-suspended into 1 ml of medium and counted using the Goryaev chamber methodology. After determining the percentage of non-settled cells, the percentage of cells remaining on the matrix was calculated (FIG. 7).

FIG. 7 shows the effect of different parameters on cell adhesion. As can be seen from the data obtained, the planting of cells in the composition of alginate gel allows for minimizing cell loss and achieving their maximum delay on the surface of the matrix. Additional treatment of matrixes with collagenase leads to an increase in adhesion in the case of the “B” sample by 64% compared to the control, the adhesion for “E” does not significantly increase, as for the rest of the samples—the adhesion either decreases slightly or does not change.

### Development of a Non-Invasive Method for Assessing Cell Proliferation

Currently, there are many methods for determining cell proliferation, all of which have their advantages and disadvantages. The methods can be divided into two main categories: the first category measures the cells, whose viability will be affected in the process of measuring their activity, and the second category is a live-cell analysis. For industrial applications, the second category is the preferred method as it allows you to evaluate proliferation in dynamics, without disrupting the technological process.

In skeletal cells, the pyruvate/lactate ratio is often used as a method for determining the metabolic activity of muscle cells. In 2016, employees of the Kurchatov Institute (GosNIIgenetics) proposed a method for determining lactic acid in biological and culture fluids of bacterial strains of lactic acid bacteria. However, due to the complex nature of these systems, the culture fluid for growing adhesive cultures of animal cells contains a great many components, including serum proteins, and a plurality of metabolites that can adversely affect the optical properties of the solution.

The spectrophotometric method makes it possible to isolate the spectrum of iron lactate and measure it separately and distinctly from the composition of the culture medium (data not shown). However, the concentration of lactate in the culture fluid of ADSCs (adipose-derived stromal cells) does not require additional dilution as described. The maximal absorption falls in the range from 380 nm to 390 nm and this range was chosen and adapted for a microplate reader.

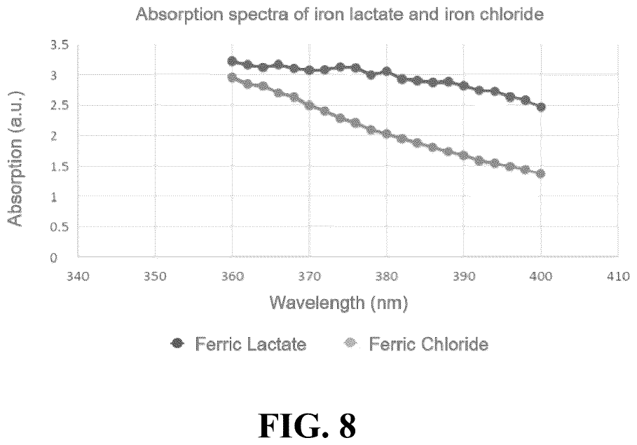

FIG. 8 shows the absorption spectra of iron lactate and iron chloride in the DMEM/F12 culture medium in a micro-plate analysis (Tecan Infinite 200 Pro) in the 360-400 nm range of the electromagnetic spectrum. As can be seen from the obtained spectra (FIG. 8), in the range from 360 nm to 400 nm, there is no overlap between the two samples, and the maximum divergence of these spectra falls in the range from 380 to 400 nm. In this region optical density measurements were to be made to ascertain the amount of lactate present. A standard curve was prepared using measurements of the optical density versus the number of cells (using a lactate measurement), which can be seen in FIG. 8.

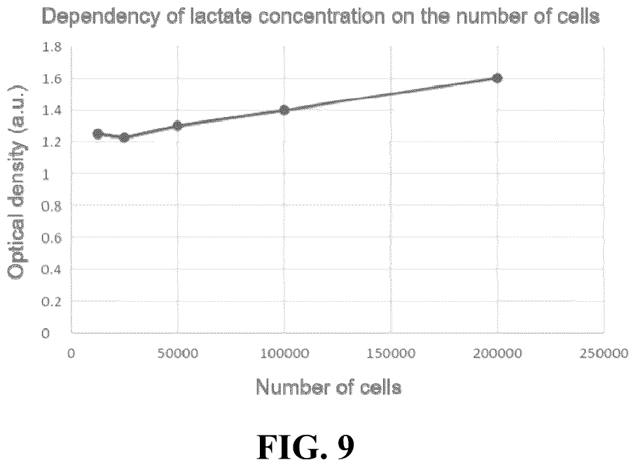

FIG. 9 shows a graph of the dependence of lactate concentration on the number of cells.

The standard curve and the sensitivity of the method were tested using the following protocol: the cells were planted using serial dilutions from 3,000 cells in a well to 200,000 cells/well. The cells were incubated in DMEM/F12 with 10% serum in a CO2 incubator for 24 hours. The data obtained allowed the determination of the minimum number of cells distinguishable by this method under the established conditions. It was determined that the minimal number of cells that could be accurately measured was 12,500 cells/ml. The results of this experiment demonstrate that this non-invasive method is not only a satisfactory method for allowing cell proliferation in bioreactors but also that the number of cells under the enumerated conditions will produce a sufficient amount of lactate for meaningful quantitative analysis.

### Isolation of the Primary Cell Line

Protocols for the isolation of myogenic and adipogenic-derived primary cells are very diverse and the present invention attempted to study the most relevant parameters that affect this isolation. The present invention combines explantation methods of isolation with enzymes. A variety of enzymes can be used in the latter technology and their activity levels are somewhat dependent on the types of biopsied tissue. The present invention opted to test a combination of collagenases and dispases. Table 3 summarizes the used methods and the concentration of the enzymes. Due to the absence of Dispase and Collagenase D, collagenase II was alone evaluated in its ability to isolate myogenic and adipogenic-derived primary cells without the use of other enzymes, while adjustments were made for its postulated activity and incubation periods.

TABLE 3
Studied methods and concentrations of enzymes used in the isolation
of primary cultures of myogenic and adipogenic tissue.

| | Collagenase I | Collagenase II | Dispase II | Collagenase D | CaCl2 |
|---|---|---|---|---|---|
| Method 1 | No | 250 CDU/ml: | 2.4 U/ml | 1.5 U/ml | 250 mM |
| | | 10 μl in 1 ml | 10 μl in 1 ml | 10 μl in 1 ml | |
| | | CaCl₂(250 mM) | CaCl₂(250 mM) | CaCl₂(250 mM) | |
| | | 30 min | a mixture is used. 60 min | | |
| Method 2 | No | No | 4.8 mg/ml | 10 mg/ml | 2.5 mm |
| | | | 2.4 U/ml | 1.5 U/ml | (110.98 |
| | | | a mixture is used. 30-40 min | | g/mol) |
| Method 3 + | 0.2% | No | No | No | No |
| chicken | (0.1 mg/ml:1.5 | | | | |
| embryo | mg per 15 ml | | | | |
| extract | PBS) 60 min | | | | |
| Method 4 | No | 0.1 mg/ml (1.5 | 1 mg/ml (15 | 1 mg/ml | No |
| (classical) | | mg to 15 ml | mg to 15 ml | | |
| | | PBS) 30 min | PBS) | | |

a mixture is used. 30 min

The present invention contemplates isolating primary cell lines using a plurality of cells. The precursors of myogenic cells can be multipotent mesenchymal stromal cells (MSCs; pluripotent cells (iPSCs, induced by various methods from fibroblasts), as well as fibro-adipogenic precursors (FAPs, that can be found in the structure of skeletal tissue) and myosatellite cells). FAP cells are of greatest interest for the cultivation of muscle and adipose tissue, as they can differentiate in the myogenic and adipogenic directions. However, the expected isolation yield of this cell type is very low, since the proportion of myosatellite cells in the body decreases greatly with age, and the cells themselves are in a stage of deep rest (in the Go phase). Given these limitations, the present invention decided to evaluate the isolation of primary cells derived from adipose tissue, such as multipotent mesenchymal stromal cells (MSCs), e.g. adipose-derived stromal/stem cells (ADSC). Moreover, adipose tissue is an ideal choice from which to isolate cells because the extraction process is minimally invasive but with great efficiency and high cell yield. In an alternate embodiment, blood and infertile tissues should be considered as options for the source of a primary cell line for cell proliferation. For example, MSCs derived from Wharton's jelly (WJ-MSC) are considered a promising source for the regeneration of various tissues, including fat and muscle. No targeted studies have been conducted for the use of WJ-MSC as a basis for the production of meat products, despite all the purported advantages.

The present invention has at least the following advantages over the prior art. The ADSCs, which are used in the present invention combine the qualities of muscle and endothelial cells; ADSCs produce extracellular matrixes and release growth factors into the media and yet can undergo more divisions, are easier and in more accessible locations to isolate, are commonly found in any tissue, and have more potencies (e.g., can be differentiated into muscle and fat tissue). In addition, compared to myogenesis, adipogenesis does not require mechanical or electrical stimulation, and the adipogenic pathways are well-defined and can be modulated with specific small molecules. Because of that, the required media composition of ADSCs is less economically demanding, thus the present invention possesses a much greater potential for significant cost reduction compared to fully cultivated meat (including muscle and endothelial cells). The present invention also contemplates using species-specific recombinant proteins, which are more ideally suited to the methods/protocols of the present invention. In addition, the matrixes used in the patent better mimic the natural ECM (extracellular matrix) of ADSC, and yet they are edible. The present invention also contemplates using edible hollow-fiber scaffold manufacturing, which will facilitate the end product formulation. Moreover, the present invention also contemplates using optimized adipogenic differentiation and lipid accumulation medium formulations to further increase the fat content, thereby solving a problem that exists in the prior art (e.g., the inability to fabricate meats that mimic real meat). The present invention also contemplates scaling up the fabrication process using conventional stirred tank reactors, which will allow for cost savings because specialized equipment is not used. Moreover, the present invention contemplates using spinner flasks with edible food-grade microcarriers even at the earliest stages of production, thereby further reducing costs.

The present invention also contemplates screening different lipid accumulation medium formulations, thereby assessing their impact on the lipid profiles of cells. The main goal of this screening is to identify and optimize formulations that will produce healthy lipid profiles, keeping the texture and taste in the 3D-structured fat closely mimicking the taste of its wild-type counterpart (i.e. from traditional animal farming).

The following protocol was performed and isolates myogenic derived primary cells from rabbit muscle tissue.

Protocol for the Isolation of Myogenic Cells from the Biopsy of Rabbit Muscle Tissue
1. Take rabbit biopsies of spinal muscle. The tissue is placed in the DMEM (complete, unenriched) in flasks and Petri dishes.
2. The flasks and Petri dishes had a 0.10% gelatin solution added to them to improve plastic adhesion and incubation was performed for 60 minutes at 37° C.
3. A solution of collagenase-II was prepared with a concentration of 0.5%.
4. The biopsy of the muscle was placed in 70% ethyl alcohol for 30 seconds for sterilization, followed by washing with sterile PBS (phosphate-buffered saline).
5. The resultant composition was centrifuged for 3 min at 200 g in a sterile PBS containing 2 equivalent concentrations of antibiotics. The supernatant was carefully removed.
6. The tissue pellet, after centrifugation, was placed in a Petri dish containing a sufficient volume of DMEM to prevent the samples from drying out during manipulation.
7. The tissue was cut into fragments of 3-5 cubic millimeters.
8. The fragments were washed and centrifuged 2 times in PBS with 1× antibiotics at 200 g for 4 minutes.
9. The pellet was resuspended in collagenase, and incubated for 15 minutes at 37° C.
10. The residual solid underwent careful rubbing of the large fragments by pipetting with a grey-throat 10 ml. wide-tipped pipette.
11. The composition was incubated for another 15 minutes at 37° C.
12. After incubation, rubbing was repeated with a 5 ml serological narrow-tipped pipette.
13. A solution of complete DMEM (or alternatively whey) was added to inactivate the enzyme, and the composition was centrifuged at 200 g for 5 min.
14. The pellet was resuspended in DMEM*, and then transferred to prepared flasks.
   *The DMEM (Dulbecco's Modified Eagle Medium) that was used for the isolation of myogenic cells: DMEM base with a high glucose content of 4.5 g/1.4 mm L-glutamine, 110 mg/1 Sodium pyruvate, 20% FBS (fetal bovine serum), and 10% HS (horse serum).

The following protocol was performed to isolate ADSCs (adipose-derived stromal cells) from rabbit adipose tissue.

Protocol for the Isolation of ADSC from Rabbit Adipose Tissue Biopsy
1. Take rabbit biopsies of adipose tissue. The biopsy specimens were placed in DMEM/F-12 complete serum, and placed in flasks and/or Petri dishes.
2. The flasks and/or Petri dishes were treated with a 0.1% gelatin solution to improve plastic adhesion and incubation was performed for 60 minutes at 37° C.
3. A preparation of collagenase-II with a concentration of 2.9 mg/ml was prepared.
4. The various biopsies of adipose tissue were placed in 70% ethyl alcohol for sterilization for 30 seconds, followed by washing in PBS solution.
5. The resulting composition was centrifuged twice at 200 g or 3 minutes in a sterile PBS containing 2 concentrations of antibiotics. The supernatant was carefully removed.
6. After centrifugation, the tissue was placed in a petri dish containing a sufficient volume of DMEM/F-12 to prevent the samples from drying out during manipulation.
7. The biopsy was cut into fragments of 3-5 cubic millimeters with sterile instruments.
8. The mixture was washed with PBS and centrifuged 2 times in the PBS with 1× antibiotics at 200 g for 4 minutes. Each time, the middle, transparent fraction was carefully removed. The white upper and lower fractions remained untouched while attempting to not lose any of the material in these fractions.
9. These fractions were resuspended in the collagenase solution and incubated for 15 minutes at 37° C.
10. The large fragments were carefully rubbed by pipetting with a 10 ml pipette.
11. Subsequently, the fragments were rubbed by pipetting with a 5 ml serological narrow-tipped pipette.
12. DMEM/F-12 was added to the composition to inactivate the enzyme, and the samples were centrifuged at 200 g for 5 minutes 3 times. Each time, the middle, transparent fraction of the supernatant was carefully removed.
13. After the last centrifugation, the precipitate was resuspended in DMEM/F-12 and then transferred to prepared flasks.
14. The samples were incubated for 24 hours. Then, the media was exchanged with fresh DMEM/F-12 to remove other cell types (e.g. erythrocytes, etc.), as well as tissue and other residues.
15. After an extra 24 hours, the cells were checked and, if necessary, another media exchange was performed.
16. After reaching 70-85% confluency, the cells were checked after ISCT criteria as well as mycoplasm contamination.

High cell yields of ADSCs were obtained from adipose tissue, with a high proliferation rate and morphological homogeneity during cell culture.

The forgoing defines and optimizes the various parameters that will give the best fabricated plant-based scaffold to generate 3D fat. Accordingly, the present invention describes embodiments for optimizing sterilization protocols, selecting and identifying the proper matrix to be used, identifying, isolating, and proliferating the various cells that should be used, identifying the composition of the serum to be used, or preferably, no serum or minimal serum, developing methodologies for enhanced proliferation, and developing a quantitative non-invasive method for ascertaining cell proliferation and/or adhesion. When these various protocols are used in combination, the 3D fat products that can be fabricated will solve many of the problems of the prior art and provide greater yields than can be attained by the presently available technology.

In an embodiment, the present invention relates to a composition comprising 3D-structured fat, wherein said composition is made by optimizing and/or using one or more of the following parameters:
a) optimizing sterilization protocols,
b) selecting and identifying an ideal matrix to be used,
c) identifying, isolating, and proliferating ideal cells to be used,
d) identifying and using animal-derived serum, or alternatively, using minimal or no serum,
e) using one or more methodologies to enhance the ideal cell proliferation, and
f) developing a quantitative non-invasive method for ascertaining the ideal cell proliferation and/or adhesion.

In a variation, the composition is made by optimizing and/or using all of the parameters. In a variation, the ideal matrix comprises a plant-based matrix. In a variation, the plant-based matrix is mainly derived from soy. By mainly, in an embodiment, it means that the ideal matrix is equal to or greater than about 60%, or alternatively, greater than about 70%, or alternatively, greater than about 80%, or alternatively, greater than about 90%, or alternatively, greater than about 95%. By minimal serum, in an embodiment, it means less than about 10%, or alternatively, less than about 5%, or alternatively, less than about 2%.

In an embodiment, the ideal cells are MSC and progenitor cells. In a variation, the progenitor cells are ADSCs and/or FAPs. In a variation, in the parameters enumerated above, or in the one or more methodologies, one or more proteases are used. In a variation, the proteases comprise one or more of a serine protease, a collagenase or a dispase. In a variation, the best serum is a serum that comprises one or more of glutathione, TGF β1, and/or FGF2. In a variation, the quantitative non-invasive method comprises measuring a quantity of lactate using a standard curve. In a variation, the ideal matrix comprises a plant-based matrix derived from soy, the ideal cells are MSCs derived from adipose tissue (ADSCs), wherein the enumerated parameters comprise using proteases, wherein the serum comprises one or more of glutathione, TGF β1, or FGF2, and wherein the quantitative non-invasive method comprises measuring a quantity of lactate using a standard curve.

In an embodiment, the present invention relates to a method of producing a 3D-structured fat, the method comprising:
a) optimizing sterilization protocols,
b) selecting and identifying an ideal matrix to be used,
c) identifying, isolating, and proliferating ideal cells to be used,
d) identifying and using animal-derived serum, or alternatively, using minimal or no animal-derived serum,
e) using one or more methodologies to enhance the ideal cells proliferation, and
f) developing a quantitative non-invasive method for ascertaining the ideal cells proliferation and/or adhesion.

In a variation, the ideal matrix is a plant-based matrix. In a variation, the plant-based matrix is derived from soy. In a variation, the ideal cells are MSC and progenitor cells, such as ADSCs and FAPs derived from adipose tissue. In a variation, the one or more methodologies comprise using proteases or the various parameter steps comprise using proteases. In a variation of the method, the proteases are selected from the group consisting of a serine protease, a collagenase, a dispase, and combinations thereof. In a variation, the best serum is a serum that comprises one or more of glutathione, TGF β1, or FGF2.

In a variation, the quantitative non-invasive method comprises measuring a quantity of lactate using a standard curve. In a variation, the standard curve is derived from a serial dilution of cells and the lactate is measured at a wavelength between 380 and 390 nm.

In a variation, the ideal matrix is derived from soy, and the ideal cells are MSCs derived from adipose tissue (ADSCs), wherein the one or more methodologies comprise using proteases, wherein the serum comprises one or more of glutathione, TGF β1, or FGF2, and wherein the quantitative non-invasive method comprises measuring a quantity of lactate using a standard curve.

In an embodiment, the present invention also relates to finding and using the best fabricated plant-based scaffold to generate 3D fat. The method involves optimizing the parameters listed above as is described in this application. For example, in a variation, the matrix that is used to generate 3D fat may be dependent upon the characteristic that one is seeking or attempting to emphasize. Matrixes that undergo degradation by a particular protease may be ideal in some circumstances. The parameters that are used and described herein are in effect tunable so that one can attain the ideal parameters/characteristics for the 3D fat one is trying to generate.

It should be recognized that because of the relatively large number of parameters that can be adjusted, the use of computers (and artificial intelligence) may prove fruitful in obtaining the best circumstances for generating 3D fat.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. Any feature that is disclosed herein can be combined with any other feature even if those features are not mentioned together, as long as those features are not incompatible. The terminology used herein was chosen to best explain the principles of the embodiments, the practical application or technical improvement over technologies found in the marketplace, or to enable others of ordinary skill in the art to understand the embodiments disclosed herein.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention. In any event, the present invention can be defined to some extent by the following claims.

We claim:

1. A composition comprising 3D-structured fat, wherein said composition is made by optimizing and/or using one or more of the following parameters:
a) optimizing sterilization protocols,
b) selecting and identifying an ideal matrix to be used,
c) identifying, isolating, and proliferating ideal cells to be used,
d) identifying and using animal-derived serum, or alternatively, using minimal or no serum,
e) using one or more methodologies to enhance the ideal cell proliferation, and
f) developing a quantitative non-invasive method for ascertaining the ideal cell proliferation and/or adhesion.

2. The composition of claim 1, wherein the composition is made by optimizing and/or using all of the parameters.

3. The composition of claim 1, wherein the ideal matrix comprises a plant-based matrix.

4. The composition of claim 3, wherein the plant-based matrix is mainly derived from soy.

5. The composition of claim 1, wherein the ideal cells are MSC and progenitor cells, such as ADSCs and FAPs.

6. The composition of claim 1, wherein one or more methodologies comprise using proteases.

7. The composition of claim 6, wherein the proteases comprise one or more of a serine protease, a collagenase or a dispase.

8. The composition of claim 1, wherein the serum comprises one or more of glutathione, TGFβ1, or FGF2.

9. The composition of claim 1, wherein the quantitative non-invasive method comprises measuring a quantity of lactate using a standard curve.

10. The composition of claim 1, wherein the ideal matrix comprises a plant-based matrix derived from soy, the ideal cells are MSCs cells derived from adipose tissue (ADSCs), wherein one or more methodologies comprise using proteases, wherein the serum comprises one or more of glutathione, TGF β1, or FGF2, and wherein the quantitative non-invasive method comprises measuring a quantity of lactate using a standard curve.

11. A method of producing a 3D-structured fat, the method comprising:
a) optimizing sterilization protocols,
b) selecting and identifying an ideal matrix to be used,
c) identifying, isolating, and proliferating ideal cells to be used,
d) identifying and using animal-derived serum, or alternatively, using minimal or no serum,
e) using one or more methodologies to enhance the ideal cells proliferation, and
f) developing a quantitative non-invasive method for ascertaining the ideal cells proliferation and/or adhesion.

12. The method of claim 11, wherein the ideal matrix is a plant-based matrix.

13. The method of claim 12, wherein the plant-based matrix is derived from soy.

14. The method of claim 11, wherein the ideal cells are MSC and progenitor cells, such as ADSCs and FAPs derived from adipose tissue.

15. The method of claim 11, wherein the one or more methodologies comprise using proteases.

16. The method of claim 11, wherein the proteases are selected from the group consisting of a serine protease, a collagenase, a dispase, and combinations thereof.

17. The method of claim 11, wherein the serum comprises one or more of glutathione, TGF β1, or FGF2.

18. The method of claim 11, wherein the quantitative non-invasive method comprises measuring a quantity of lactate using a standard curve.

19. The method of claim 18, wherein the standard curve is derived from a serial dilution of cells and the lactate is measured at a wavelength between 380 and 390 nm.

20. The method of claim 11, wherein the ideal matrix is derived from soy, the ideal cells are MSCs cells derived from adipose tissue (ADSCs), wherein the one or more methodologies comprise using proteases, wherein the serum comprises one or more of glutathione, TGF β1, or FGF2, and wherein the quantitative non-invasive method comprises measuring a quantity of lactate using a standard curve.
